(19) United States
(12) Patent Application Publication
(10) Pub. No.: US 2012/0060841 A1
(43) Pub. Date: Mar. 15, 2012

(54) OXYGEN ENRICHMENT DEVICE FOR VENTILATOR

(75) Inventors: Richard William Crawford, JR., Yucaipa, CA (US); Clayton Roy Platt, Lake Forest, CA (US)

(73) Assignee: NEWPORT MEDICAL INSTRUMENTS, INC., Costa Mesa, CA (US)

(21) Appl. No.: 12/883,021

(22) Filed: Sep. 15, 2010

(51) Int. Cl. A61M 16/12

(52) U.S. Cl. 128/205.11

(57) ABSTRACT

An enrichment device for mixing ambient air with a gas such as oxygen has a rigid outer housing defining a reservoir. The housing has an outlet port for attachment to a cyclic low pressure source, an ambient air inlet, and a second inlet for connection to a supply of pressurized gas. The reservoir has a plurality of internal walls defining a passageway having a plurality of turns defining a tortuous path for gas flow between the ambient air inlet and the outlet port, and the second inlet communicates with the passageway at a location at or close to the outlet port.

### BACKGROUND

1. Field of the Invention

The present invention relates generally to ventilators for home or hospital use for supplying gases to a patient in order to assist with their breathing, and is particularly concerned with an oxygen enrichment or gas mixing device which enriches or mixes air supplied to the ventilator inlet with oxygen or other gases at a selected ratio.

2. Related Art

Patients suffering from various diseases such as chronic respiratory diseases, spinal chord injuries, sleep apnea, and the like require complete or partial assistance with breathing. In some cases, breathing must be completely taken over by a ventilator. In other cases, a patient needs only partial support of their normal breathing In the latter case, a patient's normal breathing function can be supported partially by a ventilation system known as pressure support breathing. Ventilators which partially support a patient's breathing can be used in hospitals or in the home. Such ventilators typically include a gas mixer or oxygen enrichment device which mixes oxygen with air for supply to a patient through the ventilator, with the ratio of oxygen to air varying dependent on the specific patient requirements.

In some home use ventilators, oxygen blending is provided by an oxygen blending bag attached to the air inlet port of the ventilator. However, the oxygen blending bag is not a calibrated oxygen mixing device and requires use of an oxygen monitor to verify the level of oxygen enrichment. Other gas blenders for home or hospital ventilators used in assisting a patient's breathing use proportioning systems to maintain accurate blending of atmospheric air and pressurized gas such as oxygen, but such systems are relatively complex and involve parts such as control valves which move during operation of the device. Such systems are therefore relatively expensive and require frequent maintenance.

### SUMMARY

Embodiments described herein provide for an oxygen enrichment device for supplying a mixture of air and oxygen or other gases at a selected ratio to a ventilator.

According to one embodiment, an enrichment device for mixing ambient air with a gas has a rigid outer housing defining a reservoir and having an outlet port for attachment to a cyclic low pressure source, an ambient air inlet, and a second inlet for connection to a supply of pressurized gas. The reservoir has a passageway for gas flow between the ambient air inlet and the outlet port, and the second inlet communicates with the passageway at a location at or close to the outlet port. The housing contains no parts which move during operation of the device. The passageway may have a restricted inlet portion extending from the ambient air inlet along part of the passageway which is configured to control air flow rate into the reservoir. In one embodiment, the housing has a plurality of internal walls or baffles forming the passageway which define a path for gas through the housing which has a plurality of turns. Both the restricted inlet portion and the tortuous or winding path through the housing formed by the plural turns in the passageway help to control the ratio of gas to ambient air drawn out of the reservoir through the outlet port, and the device has no parts which are required to move during operation of the device in order to control gas mixing.

Air is drawn into the restricted inlet portion of the passageway and a mixture of gas and air is drawn out of the reservoir through the outlet port when the cyclic low pressure source is on, and pressurized gas fills at least part of the passageway when the low pressure source is off. Variation of the flow rate of gas from the pressurized source into the housing varies the ratio of gas to ambient air in the mixture drawn out of the reservoir through the outlet ports. In one embodiment, the outlet port is connected to a ventilator inlet, so that the gas and air mixture is drawn out of the reservoir when a ventilator pump is turned on, i.e. when a patient connected to the ventilator takes a breath, and gas starts to fill the passageway between patient breaths when the pump is off, with the flow rate of pressurized gas controlling how much gas enters the reservoir between breaths and thus the ratio of gas to ambient air drawn out of the reservoir when the pump is on.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is an exploded perspective view of one embodiment of an oxygen enrichment or gas mixing device;

FIG. 2 is vertical cross-sectional view of the oxygen enrichment device of FIG. 1 in an assembled condition;

FIG. 3 is a horizontal cross-section on the lines 3-3 of FIG. 2;

FIG. 4 is a cross-sectional view similar to FIG. 2 but showing the gas flow path through the device;

FIG. 5 is a functional block diagram showing the oxygen enrichment device connected to a ventilator and pressurized oxygen supply; and

FIG. 6 is an example of a graph illustrating oxygen supply flows corresponding to desired percentage of oxygen enrichment using the oxygen enrichment device of FIGS. 1 to 3.

### DETAILED DESCRIPTION

Certain embodiments as disclosed herein provide for an oxygen enrichment or gas mixing device for a ventilator suitable for home or hospital use in assisting a patient's breathing. Although the following description concerns blending of oxygen at desired percentage levels with ambient air, it will be understood that the device may alternatively be used for mixing different gases together at a controlled ratio.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention.

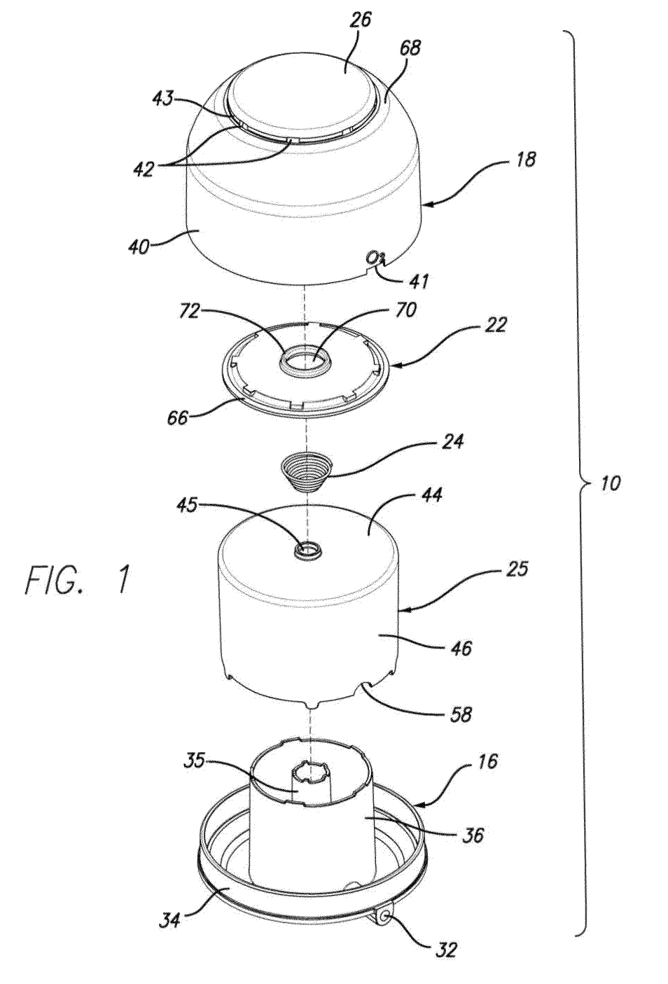

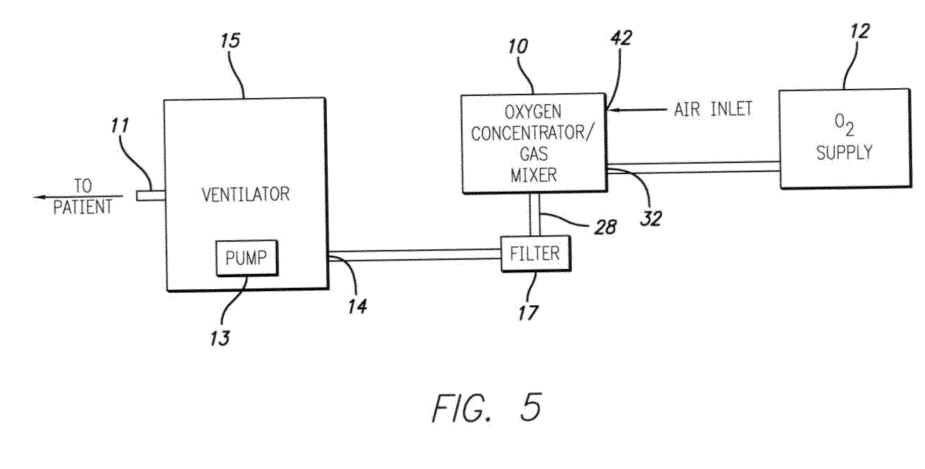

FIGS. 1 to 4 illustrate one embodiment of a gas mixing or oxygen enrichment device 10, while FIG. 5 illustrates connection of the device 10 to a pressurized oxygen supply 12 and to the inlet or gas intake port 14 of a ventilator 15. Ventilator 15 has a standard outlet connection 11 for connection to a patient delivery tube or conduit. An optional removable filter 17 may be located between the oxygen concentrator outlet 28 and the ventilator gas inlet 14, to remove particulates from the oxygen/air mixture flowing from the reservoir chamber within device 10. The oxygen enrichment device may be used with any pressure support ventilator designed for home use, such as the HT50 Ventilator manufactured by Newport Medical Systems Inc. of Newport Beach, Calif. The ventilator has a cyclic low pressure pump 13 which is actuated when a patient takes a breath and is turned off between breaths.

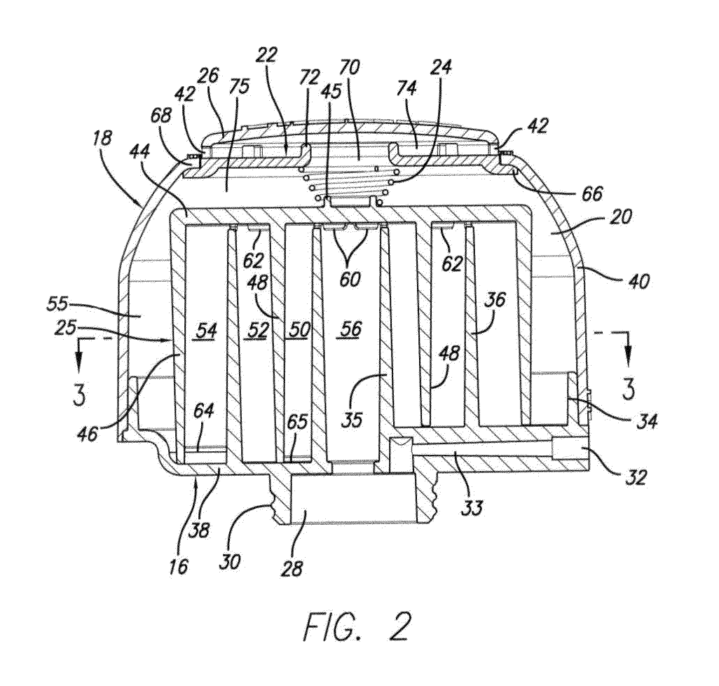

As illustrated in FIGS. 1 and 2, the oxygen enrichment device 10 basically comprises an outer housing of rigid material defining a reservoir for air and oxygen. The housing has a base 16, a hard outer shell or cover 18 of rigid plastic or metal designed for releasable attachment to the base to form a reservoir or gas mixing chamber 20 within the outer shell, a baffle plate 22, a spring 24, and a base baffle 25 contained in the outer shell 18 and secured in the reservoir or mixing chamber between first and second end walls 26 and 38. In the following description, the first end wall is defined as an upper end wall while the second end wall is defined as a lower end wall. However, although the device may be oriented vertically with the first end wall uppermost in some embodiments, it should be understood that it may be oriented in different directions in alternative embodiments, including horizontal orientations. The terms “upper” and “lower” in the following description should not be interpreted as limiting the housing to use in vertical orientations, and are used for convenience only in the following description.

Base 16 and base baffle 25 have interleaved cylindrical walls or baffles when the device is assembled as in FIG. 2, and the base and base baffle, together with baffle plate 22, provide internal walls in the reservoir which together form a winding passageway for air or oxygen through the reservoir. The passageway has a plurality of bends forming a tortuous path for incoming air and oxygen flowing into the reservoir chamber, as described in more detail below. The number of turns or bends in the passageway and the passageway dimensions, together with the controlled flow rate of oxygen into the housing, control the ratio of oxygen and air drawn out of the device 10 in each patient breath, as described in more detail below. The passageway is of serpentine shape along at least part of its length in the illustrated embodiment, but other tortuous passageway shapes with multiple bends may be used in other embodiments.

Base 16 has a central outlet port 28 which has external threads 30 for screw connection to the ventilator inlet port, an oxygen inlet 32, an annular outer rim 34, and inner and outer concentric, upwardly facing cylindrical walls or tubes 35, 36 extending upwardly from the lower wall 38 of the base. The inner tube or cylindrical tube 35 defines a central conduit 56 which communicates with the outlet port 28. As illustrated in FIG. 2, the oxygen inlet 32 is also connected to the gas outlet port 28 via passageway 33. The reservoir top shell or cover 18 has an outer wall 40 designed to engage over the outer rim 34 of the base and has an indent 41 on its lower edge which engages over the oxygen inlet 32 in the base. Ambient air inlet ports 42 are provided at spaced intervals around the indented outer rim 43 of the cover top wall 26. A filter such as a removable filter (not illustrated) similar to those used in NATO gas masks may be attached over the ambient air inlet ports 42 to filter incoming air.

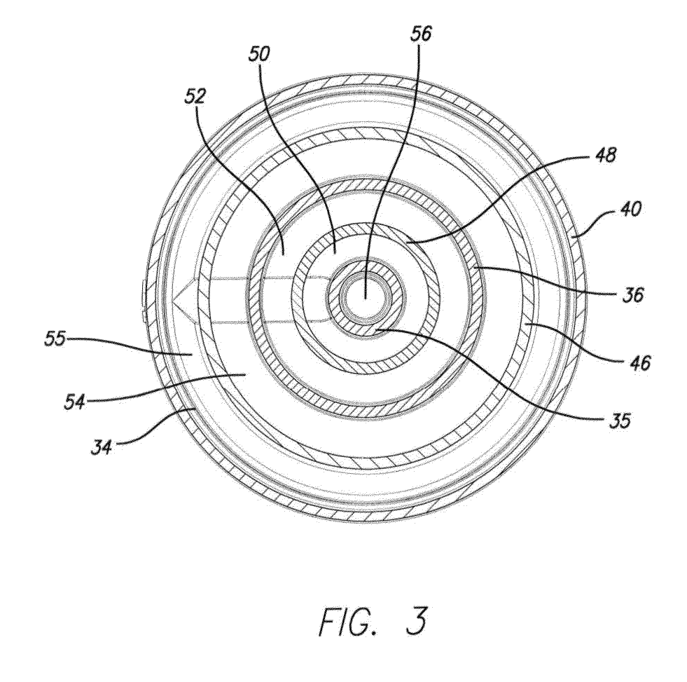

The reservoir base baffle 25 is of inverted cup-like shape with an upper wall 44 having a central raised rim or spring seat 45, an outer cylindrical wall 46 of diameter less than that of the cover 18, and an inner cylindrical baffle or wall 48 which has a diameter greater than that of the inner cylindrical base baffle 35 and less than that of the outer cylindrical base baffle 36. The arrangement is such that, when the reservoir base baffle 25 is telescopically engaged over the outer cylindrical wall 36 of the base 16, the outer cylindrical baffle wall 46 engages over the outer cylindrical base wall 36, while the inner cylindrical baffle wall 48 engages over the inner cylindrical base wall 35, as best illustrated in FIG. 2. Thus, the cylindrical walls 35, 48, 36, 46 and cylindrical lower portion of wall 40 are interleaved concentrically to form a series of annular passageways or passageway portions 50, 52, 54 and 55 of gradually increasing diameter extending outwardly from the central conduit 56 defined by inner cylindrical base wall 35, as illustrated in FIGS. 2 and 3. The base baffle and base together provide a torturous or winding flow path from the air inlet to the central passageway 56, and from the oxygen inlet outwardly from the central passageway, which improves gas ratio precision.

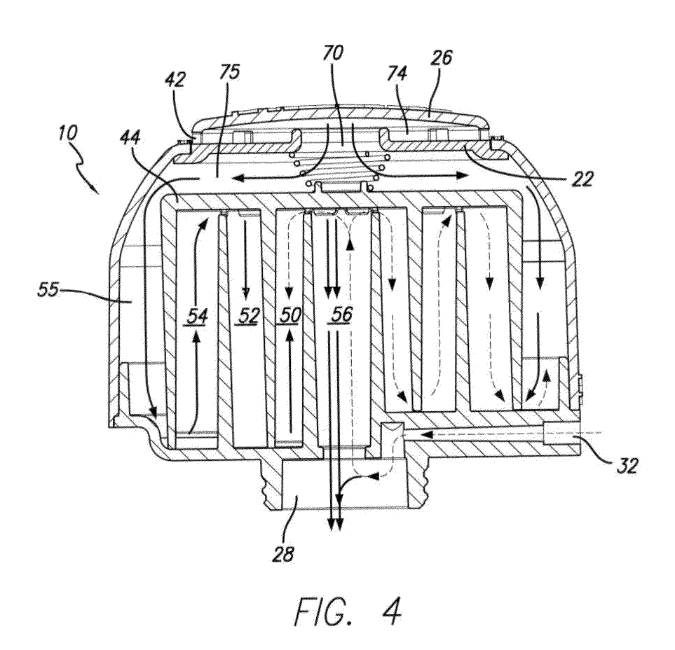

As best illustrated in FIG. 1, the upper ends of both the inner and outer cylindrical base walls 35 and 36 are castellated to form a series of alternating protrusions and indentations. The lower ends of the outer and inner baffle walls 46 and 48 of the base baffle are also castellated, as illustrated in FIGS. 1 and 2, and the inner and outer baffle walls also each have a rounded indent 58 designed to engage over the wall forming passageway 33, as best illustrated in FIG. 2. The castellated protrusions on the upper ends of the inner and outer cylindrical base walls engage the inner face of the upper wall 44 of base baffle 25 while restricted passageways or openings 60, 62 are formed between adjacent protrusions of the inner cylindrical base wall 35 and outer cylindrical base wall 36, respectively, as illustrated in FIG. 2. Openings 60 allow gas flow between the upper ends of inner annular passageway 50 and central tubular passageway 56, while openings 62 allow gas flow between the upper ends of annular passageways 54 and 52. Similarly, the castellated protrusions on the lower ends of the outer and inner baffle walls 46, 48 engage the inner face of base end wall 38 to define restricted passageways or openings 64 and 65 between adjacent protrusions of the outer and inner baffle walls, respectively. Openings 64 allow gas flow between the lower ends of outer annular passageway 55 and adjacent annular passageway 54, while openings 65 allow gas flow between the lower ends of annular passageways 52 and 50. Gas flow and mixing is described in more detail below in connection with FIG. 4.

As best illustrated in FIG. 2, baffle plate 22 is located between the upper wall 26 of top cover 18 and the upper wall 44 of base baffle 25, and has an outer annular rim 66 which engages under an annular shoulder or stop portion 68 of the cover outer wall 40, and a central opening 70 with an upturned outer rim 72 projecting upwards towards the cover upper wall 26. Spring 24 is located between the upper wall 44 of base baffle 25 and the baffle plate 22 to hold the baffle plate in the position illustrated in FIG. 2. Baffle plate 22 defines a restricted air inlet portion 74 between plate 22 and upper wall 26 for air flowing into the device via air inlets 42. The spring engages over the central locating rim 45 on the upper wall 44 of the base baffle, and helps to keep baffle plate 22 and base baffle 25 in the correct position.

FIG. 4 illustrates the flow paths for incoming pressurized oxygen via inlet port 32 (dotted lines with arrows), incoming ambient air via inlets 42 (solid lines with arrows), and the flow of air and oxygen mixed in reservoir 20 out of the device gas outlet port 28 for supply to a ventilator gas inlet port (double lines with arrows). Although the air path is shown primarily on the left half of the device in FIG. 4 while the oxygen path is shown primarily on the right half, it should be understood that air and oxygen each flow equally through the entire circumference of each annular passageway to the extent determined by the oxygen flow rate, the time between patient breaths, the passageway dimensions and number of bends.

The outer housing, baffle plate 22, base baffle 25, and cylindrical walls of the base together form the passageway for air through the housing, with the passageway having a plurality of turns forming a tortuous path for both air and incoming oxygen through the reservoir. Air flows in through inlets 42, through restricted inlet portion 74 of the passageway between the upper wall 26 and the baffle plate 22, through the central opening 70 in the baffle plate, and then outwardly through the space 75 between the baffle plate 22 and upper wall 44 of the base baffle and down through outer annular portion 55 of the passageway. Oxygen flows in through inlet 32 and passageway 33 to the port 28, as illustrated by the dotted lines, flowing upwardly through central conduit 56 when the ventilator pump is not operating (i.e. between patient breaths), then through openings 60 into the adjacent annular passageway portion 50, down to the lower end of portion 50, then outwardly through openings 65 into the next annular passageway portion 52, and so on. Thus, air flows inwardly up and down through the successive annular portions of the passageway formed by the interleaved baffles, while oxygen flows outwardly and up and down through the same passageway portions. When a breath is taken, the ventilator pump 13 is actuated and starts to extract air and oxygen from the reservoir and into the ventilator inlet system. The incoming oxygen is under pressure, so incoming oxygen displaces ambient air in the reservoir between breaths, when the ventilator pump is off. The higher the pressure and flow rate of the oxygen, the more ambient air it displaces between patient breaths. The ratio of oxygen to air in the mixture supplied to the ventilator is dependent on how much oxygen flows into the reservoir between breaths, which is controlled by the passageway dimensions, the number of bends, and the oxygen flow rate. The air and oxygen mixes as it is withdrawn from the reservoir through passageway 56 and outlet port 28, and continues to mix through the ventilator pump assembly and breathing circuit.

The interface between the top wall 26 of the outer shell and the baffle plate 22 creates a restricted inlet portion 74 for ambient air entering the reservoir. This creates a resistance to air flow which helps to provide a more precise ratio of oxygen to ambient air in the mixture supplied to the ventilator. The number of bends in the path also helps to control the accuracy of the oxygen to air ratio. In the illustrated embodiment, there are six bends in the passageway between air inlets 42 and outlet 28. A greater or lesser number of bends may be provided in alternative embodiments, for example by providing a lower or higher number of interleaved cylindrical walls on baffles. In alternative embodiments, passageways with three, four or more bends may be provided. The base baffle walls interleaved with the upstanding cylindrical base walls of the base 16 create a restricted, torturous path through the reservoir which inhibits and directs the flow of combined gases within the sealed device 10, further controlling the amount of air and oxygen flowing into the passageway and the ratio of oxygen to ambient air.

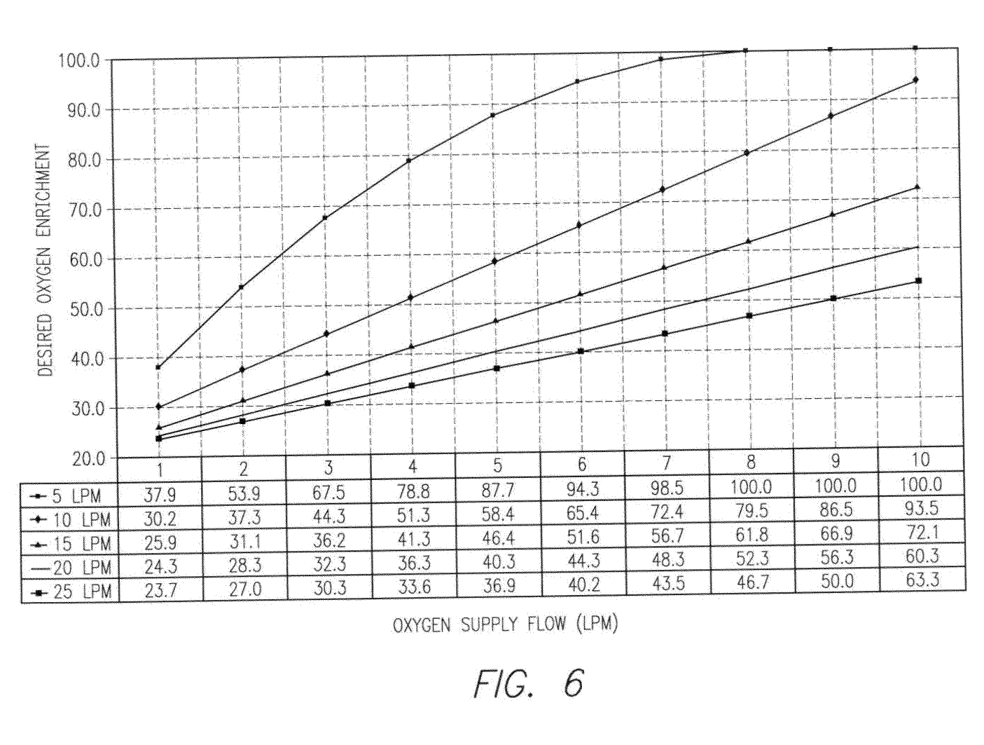

The ratio of oxygen to air supplied by device 10 can vary between 21 to 100%, with 21% being ambient air and 100% being oxygen only. The ratio is varied by manual adjustment of the oxygen flow rate according to an oxygen enrichment flow graph or table which can be created by suitable calibration of the device attached to a ventilator, as is known in the field for prior art gas mixing or oxygen concentration devices. FIG. 6 is an example of an oxygen enrichment flow graph without PEEP (positive end-expiratory pressure) for one embodiment of the oxygen concentrator device of FIGS. 1 to 4. In order to determine the proper oxygen flow rate setting, the desired percentage of oxygen enrichment is first selected. The desired setting is then followed horizontally until it meets with the line which is equal to the minute volume of the patient (i.e. 5 LPM, 10LPM, etc.). The point on the selected line is then followed down vertically until it meets the estimated oxygen supply flow (LPM) between 0 and 10 LPM. For example, if a delivered minute flow to the patient of 10 LPM with an oxygen percentage of 70% is desired, the oxygen flow rate is set to about 6.5 LPM. It should be noted that the x and y axes of the graph may be reversed, with desired oxygen enrichment as the horizontal or x axis rather than the y axis as illustrated in FIG. 6. A similar graph is used for ventilating with PEEP (positive end-expiratory pressure), with the device suitably calibrated for desired patient minute volume to produce the chart or graph. The table below the graph shows oxygen enrichment percentages for different patient delivered minute volumes and oxygen supply flow settings, e.g. for a patient minute volume of 10 LPM and an oxygen supply flow of 7, the oxygen percentage in the supplied gas mixture is around 72.4%.

Although the oxygen enrichment device described above is not as accurate as some more complex devices including moving parts such as valves and the like, it is much simpler in construction and requires less maintenance than devices with moving parts. It is also more accurate and more durable than a simple oxygen blending bag as used in the past. The restricted openings and passageway through the reservoir chamber are machined to precise dimensions to provide metered flow of gases through the chamber. The passageway dimensions together with the multiple bends in the gas flow path control the oxygen and air ratios to a relatively high level of precision without requiring moving parts, other than the biasing spring.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

We claim:

1. An enrichment device for mixing ambient air with a gas, comprising:
an outer housing defining a reservoir, the outer housing having first and second end walls and a peripheral wall, the housing having an outlet port for attachment to a cyclic low pressure source, an ambient air inlet, and a second inlet for connection to a supply of pressurized gas;
the reservoir having a plurality of internal walls defining a passageway between the ambient air inlet and the outlet port, the passageway having a plurality of turns and defining a flow path for air and gas through the reservoir; and
the second inlet communicating with the passageway downstream of the ambient air inlet;
whereby air is drawn into the passageway and a mixture of gas and air is drawn out of the reservoir through the outlet port when the low pressure source is on, and pressurized gas fills at least part of the passageway when the low pressure source is off, and variation of the flow rate of gas from the pressurized source into the housing varies the ratio of gas to ambient air in the mixture drawn out of the reservoir through the outlet port.

2. The device of claim 1, wherein the passageway has a restricted inlet portion extending from the ambient air inlet along part of the passageway which is configured to control air flow rate into the reservoir, the second inlet communicating with the passageway downstream of the restricted inlet portion.

3. The device of claim 1, wherein the second inlet communicates with the passageway at the outlet port.

4. The device of claim 1, wherein the passageway has at least four turns.

5. The device of claim 1, wherein the passageway is of serpentine shape along at least part of its length.

6. The device of claim 1, wherein the housing has a central axis and a plurality of cylindrical walls of increasing diameter are mounted in the reservoir between the central axis and peripheral wall of the housing to define successive annular portions and a central tubular portion of the passageway, the turns in the passageway including u-turns located alternately adjacent the respective end walls of the housing between successive annular portions and between an innermost annular portion and the central tubular portion.

7. The device of claim 1, wherein the housing has a central axis between the first and second end walls, the outer peripheral portion of the housing has a circular cross-section along at least part of its length, and the passageway includes portions extending radially inwardly and outwardly in the reservoir.

8. The device of claim 7, wherein the outlet port comprises a central port in the second end wall, and the passageway further includes successive annular portions of decreasing diameter and a central conduit which communicates with the outlet port.

9. The device of claim 8, wherein the ambient air inlet communicates with a portion of the passageway adjacent the first end wall.

10. The device of claim 9, wherein the second inlet communicates with the outlet port.

11. The device of claim 1, wherein the gas is oxygen and the device is an oxygen enrichment device.

12. The device of claim 1, wherein the outlet port is a central port in the second end wall of the housing, and the air inlet is located at or adjacent the first end wall of the housing.

13. The device of claim 12, wherein internal walls comprise at least a central tubular wall defining a central conduit communicating with the outlet port and a plurality of cylindrical walls spaced outwardly from the central tubular wall between the central tubular wall and the outer peripheral wall of the housing to define a plurality of annular portions of the passageway between the first and second end walls of the housing.

14. The device of claim 13, wherein the central tubular wall and cylindrical walls have first ends which are spaced from the first end wall of the housing and second ends at the second end wall of the housing, and a transverse wall extends across the first ends of the central tubular wall and cylindrical walls, the central tubular wall and cylindrical walls having openings which alternate between first and second ends of the respective walls for communication between successive passageway portions and the central conduit.

15. The device of claim 14, wherein the second inlet communicates with the lower end of the central conduit.

16. The device of claim 14, wherein the central tubular wall extends upwardly from the lower wall of the housing and at least a first cylindrical wall of larger diameter than the central tubular wall extends from the second end wall towards the first end wall, the first end of each wall engaging the transverse wall, and at least a second cylindrical wall extends from the transverse wall towards the second end wall between the central tubular wall and first cylindrical wall, the second end of the second cylindrical wall engaging the second end wall of the housing.

17. The device of claim 16, wherein the transverse wall has an outer periphery spaced inwardly from the outer peripheral wall of the housing and a third cylindrical wall extends from the outer periphery of the transverse wall to the second end wall of the housing, the outer peripheral wall, third, first and second cylindrical walls, and central tubular wall together forming successive first, second, third and fourth annular portions of the passageway from the air inlet to the central conduit, openings at the second ends of the third and second cylindrical walls providing communication between the first and second annular portions of the passageway and the third and fourth annular portions of the passageway, respectively, and openings at the first ends of the first cylindrical wall and central tubular wall providing communication between the second and third annular portions of the passageway and between the fourth annular portion and central conduit of the passageway, respectively.

18. The penetrator of claim 17, wherein the transverse wall and second and third cylindrical walls are formed integrally as a base baffle member separate from the second end wall of the housing, and a biasing mechanism acts on the transverse wall to bias the baffle member against the second end wall of the housing.

19. The penetrator of claim 14, further comprising a baffle plate between the transverse wall and upper wall of the housing which has a central opening and defines a restricted inlet portion of the passageway, whereby incoming air flows in a path which extends radially inward along the restricted inlet portion, through the central opening in the baffle plate and radially outward between the baffle plate and transverse wall before entering an annular portion of the passageway.

20. The device of claim 17, wherein the first ends of the central tubular wall and first cylindrical wall portion and the second ends of the second and third cylindrical wall portions are castellated to form said openings.

21. The device of claim 1, wherein the outer housing comprises a base including the first end wall of the housing and a rigid outer cover secured to the base, and a base baffle is contained in the housing between the first and second end walls, the base and base baffle having interleaved cylindrical walls forming at least part of said passageway.

22. The device of claim 21, further comprising a biasing mechanism in the housing between the first end wall and base baffle which biases the base baffle into engagement with the second end wall of the housing.

23. The device of claim 22, wherein the base baffle has a transverse wall engaged by the biasing mechanism and at least two concentric, cylindrical walls of first and second different diameters extending from the transverse wall to the second end wall of the housing.

24. The device of claim 23, wherein the base has at least two concentric, cylindrical walls of third and fourth diameters extending from the second end wall to the transverse wall, the third diameter being less than the first diameter and the fourth diameter being between the first and second diameters, whereby the cylindrical walls are interleaved to form annular portions of the passageway, and the smallest diameter wall of the base forms a central outlet conduit which communicates with the outlet port.

25. The device of claim 22, further comprising a transverse baffle plate between the base baffle and upper wall of the housing, the biasing mechanism acting between the baffle plate and base baffle.

26. The device of claim 25, wherein the air inlet comprises a plurality of openings in the outer cover between the baffle plate and first end wall of the housing, a gap defined between the baffle plate and upper wall defining a restricted inlet portion of the passageway, and the baffle plate has a central opening providing communication between the restricted inlet portion and remainder of the passageway through the reservoir.

27. The device of claim 1, further comprising a removable filter at the outlet port which is configured to filter particulates from the gas mixture drawn out through the outlet port.

28. The device of claim 1, further comprising a filter at the ambient air inlet which filters ambient air drawn into the reservoir through the air inlet.

29. An enrichment device for mixing ambient air with a gas, comprising:
an outer housing of rigid material defining a reservoir, the outer housing having first and second end walls and a peripheral wall, the housing having an outlet port for attachment to a cyclic low pressure source, an ambient air inlet located at or adjacent the first end wall and spaced from the outlet port, and a second inlet for connection to a supply of pressurized gas;
the reservoir having a passageway defining a path for gas flow between the ambient air inlet and the outlet port; and
the second inlet communicating with the passageway downstream of the ambient air inlet;
the housing containing no parts which move during operation of the device;
whereby air is drawn into the passageway and a mixture of gas and air is drawn out of the reservoir through the outlet port when the low pressure source is on and pressurized gas fills at least part of the passageway when the low pressure source is off, and variation of the flow rate of gas from the pressurized source into the housing varies the ratio of gas to ambient air in the mixture drawn out of the reservoir through the outlet port.

30. The device of claim 29, further comprising internal walls in the reservoir forming the passageway between the ambient air inlet and the outlet port.

31. The device of claim 30, wherein the internal walls define a tortuous path from the air inlet to the outlet port, the path having a plurality of bends.

32. The device as claimed in claim 31, wherein the path has at least three bends.

33. The device as claimed in claim 32, wherein the path has six bends.

34. The device of claim 31, wherein the path includes first portions extending parallel to the end walls and second portions extending in directions transverse to the first portions along at least part of the length of the reservoir and back and forth between the first and second end walls.

35. The device of claim 29, wherein the passageway includes a restricted inlet portion of reduced dimensions extending from the air inlet, the restricted inlet portion controlling the flow rate of incoming ambient air.

36. The device of claim 29, wherein the outlet port is located in the second end wall.

37. The device of claim 36, wherein the second inlet communicates with the passageway adjacent the outlet port.

38. A ventilator system, comprising:
a ventilator having a gas inlet, an outlet for connection to a patient breathing gas delivery tube, and a cyclic low pressure pump which is actuated when a patient takes a breath;
an oxygen enrichment device having a reservoir, an outlet port connected to the ventilator gas inlet, at least one ambient air inlet, and an oxygen inlet, a plurality of internal walls in the reservoir defining a tortuous, winding passageway from the ambient air inlet to the outlet port which has a plurality of bends, and the oxygen inlet communicating with the passageway and outlet port; and
a pressurized oxygen supply connected to the oxygen inlet of the enrichment device, the oxygen supply having an adjustable flow rate;
whereby adjustment of the flow rate of oxygen to the oxygen enrichment device varies the ratio of oxygen and ambient air in the gas mixture supplied from the enrichment device to the ventilator when the pump is actuated.

39. The system of claim 38, wherein the passageway in the oxygen enrichment device has a restricted inlet portion extending from the ambient air inlet along part of the passageway which is configured to control air flow rate into the reservoir, the second inlet communicating with the passageway downstream of the restricted inlet portion.

40. The system of claim 39, wherein the second inlet communicates with the passageway at the outlet port.

41. The system of claim 38, wherein the passageway has at least four bends.

42. The system of claim 38, wherein the passageway is of generally serpentine shape along at least part of its length.

43. The device of claim 38, wherein the oxygen enrichment device comprises a rigid outer housing having a first end wall, a second end wall, and an outer peripheral wall, and plurality of cylindrical walls of different diameters in the reservoir between the first and second end walls forming annular portions and a central conduit of said passageway.

44. The device of claim 43, wherein the second end wall has a central opening which communicates with the outlet port.

45. The device of claim 44, wherein the ambient air inlet is located at or adjacent the first end wall of the housing and at least a first transverse wall spaced from the first end wall forms a restricted air inlet portion of the passageway, the transverse wall having a central opening.

46. The device of claim 45, further comprising a second transverse wall spaced from said first transverse wall, the internal cylindrical walls extending from said second transverse wall to the first end wall of the housing.

47. The device of claim 46, wherein at least two concentric, inner and outer cylindrical walls extend between the second end wall and second transverse wall, the inner cylindrical wall defining the central conduit and the outer cylindrical wall defining annular portions of the passageway.

48. The device of claim 43, wherein the air inlet comprises a plurality of openings in the outer housing.

49. A method of mixing ambient air with gas from a pressurized gas source for supply to a ventilator gas inlet port, comprising:
periodically actuating a cyclic low pressure source connected to an outlet port at one end of a rigid housing to draw ambient air into the housing through an air inlet port at or adjacent an opposite end of the housing and along at least part of a passageway in the housing between a first end at the air inlet and a second end at the outlet port;
supplying pressurized gas to the second end of the passageway;
whereby ambient air travels along the passageway from the first end when the low pressure source is actuated and pressurized gas travels along the passageway from the second end when the low pressure source is off, and a mixture of pressurized gas and air is drawn out of the outlet port when the low pressure source is actuated, the ratio of pressurized gas to air depending on the amount of pressurized gas in the passageway when the low pressure source is switched from off to on; and
adjusting the flow rate of pressurized gas into the second end of the passageway to vary the ratio of gas to ambient air in the mixture drawn out of the outlet port.
